(19) United States
(12) Patent Application Publication
(10) Pub. No.: US 2006/0149318 A1
(43) Pub. Date: Jul. 6, 2006

(54) Ergonomic ped-hydro cavity agitation for therapeutic assistance and relaxation use

(76) Inventor: Kevin Ballentine, Baltimore, MD (US)

Correspondence Address: KEVIN BALLENTINE, 2760 KINSEY AVENUE, BALTIMORE, MD, 21223, US

(21) Appl. No.: 11/029,202

(22) Filed: Jan. 4, 2005

(51) Int. Cl. A61N 1/39; A61K 31/045

(52) U.S. Cl. 607/1; 514/738

(57) ABSTRACT

This invention is an ergonomic device that has been created with the notion of removing barriers to quality, productivity, and safe human performance by fitting products, tasks, and environments to people. The invention is designed to provide a service to people who are concerned about the long term problems that can occur from excessive muscle use while performing strenuous tasks and utilizing innovative technological apparatuses. It is a therapeutic good that has the ability to provide assistance for people with musculoskeletal disorders, occupational overuse syndrome, fatigue, and injuries related to the vascular system or the nervous system. The invention is an ergonomically created foot wear, worn while resting, that has the ability to provide relaxation, reduce swelling, induce blood flow, and rejuvenate the muscles and nerves in at least the ankle and foot area.

### BACKGROUND OF THE INVENTION

(1) Technical Field

This invention pertains to the field of ergonomics. It has been created with the notion of removing barriers to quality, productivity, and safe human performance by fitting products, tasks, and environments to people. The invention is designed to provide a service to people who are concerned about the problems that can occur from excessive muscle use while performing strenuous tasks and utilizing innovative technological apparatuses.

(2) Description of the Related Art

According to the United States Census Bureau, “Musculoskeletal disorders account for nearly 70 million physician office visits in the United States annually and an estimated 130 million total health care encounters including outpatient, hospital, and emergency room visits.” The primary objective of this invention is to help assure the safety of those people that possess high risk of musculoskeletal disorders and/or occupational overuse syndrome related to repetitious movements and innovative technology.

### BREIF SUMMARY DESCRIPTION OF THE INVENTION

The invention was designed to provide support, assistance, and comfort for those people that possess musculoskeletal disorders and/or occupational overuse syndrome; related to repetitious movements and innovative technology. It is an ergonomically created foot wear, worn while resting, that has the ability to provide relaxation, reduce swelling, induce blood flow to muscles, and rejuvenate the nerves within desired areas of the foot. The invention has the ability to stimulate the blood vascular system and nervous system to help reduce stressors and enhance healing processes within the anatomy of the body, such as the neck, back, arms, shoulders, liver, lungs, brain and heart.

### BRIEF DESCRIPTION OF THE SEVERAL VIEWS OF THE DRAWING

FIG. 1 displays the bottom closure casing for the agitation system. The drawing displays two ports, 14 and 16, that grip the oscillation motor systems, 21 and 22, that provoke agitation to the fluid cavity system of FIG. 4. Located in the center of the bottom closure covering is a circular arch that extends through holes 24 and 37. The inside of the bottom closure is ergonomically shaped to hold all individual parts necessary securely. Placed on the opposite side of the bottom closure case are gripping 46 for prevention of slipping when place on various floor surfaces. On the opposite side is an enclosure port 23 used for storing the power supply of the fluid cavity agitation system.

FIG. 2 displays the fluid cavity agitation system. The system activates agitation within the cavity pockets (FIG. 4) to induce oscillation of the non-toxic antifreeze fluid.

FIG. 3 matches a reflexology diagram that indicates the pressure points of the foot known to stimulate the vascular system and nerves system to the ergonomic top covering of the agitation system. The agitation system top covering has small circular ridge arches 38,39,40,41,42,43,44,45 that are placed in specified areas to stimulate nerve endings within the lower extremities the foot.

FIG. 4 displays the fluid cavity pocket system. In it are holes 23, 24, and 25 that allow the fluid cavity pocket system to fit firmly together with FIG. 1 and FIG. 3. Fluid cavities 26, 27, 28,30,31,32,33,34,35, and 36 hold a non toxic anti-freeze fluid.

FIG. 5 displays protective closure covering for the invention. The covering is made from elastic plastic material that protects the fluid cavity pocket system from being punctured or damaged.

FIG. 6 is a composite of all parts of the invention. It displays how FIG. 1, FIG. 2, FIG. 3, FIG. 4, and FIG. 5 are combined together to make the invention.

FIG. 7 displays the posture of a foot while in a relaxing state. It displays the main arch 51, instep 53, profile 54 and vamp 55. The ball of the foot is indicated as number 50 and the back arch/heel area is indicated as 52.

### DETAILED DESCRIPTION OF THE INVENTION

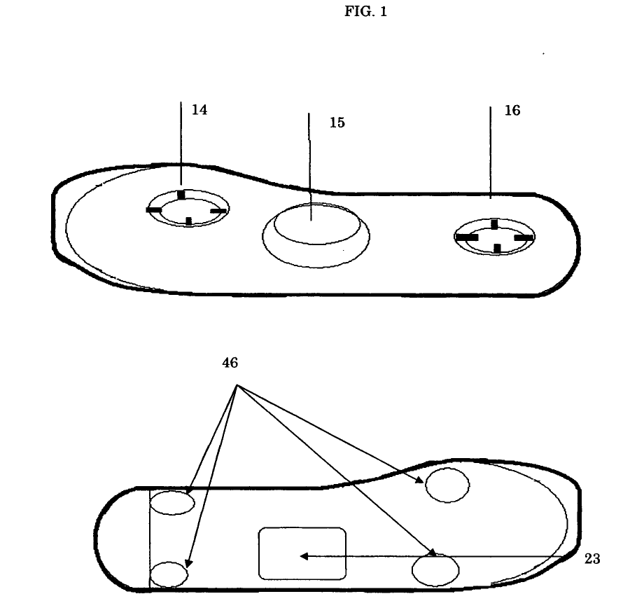

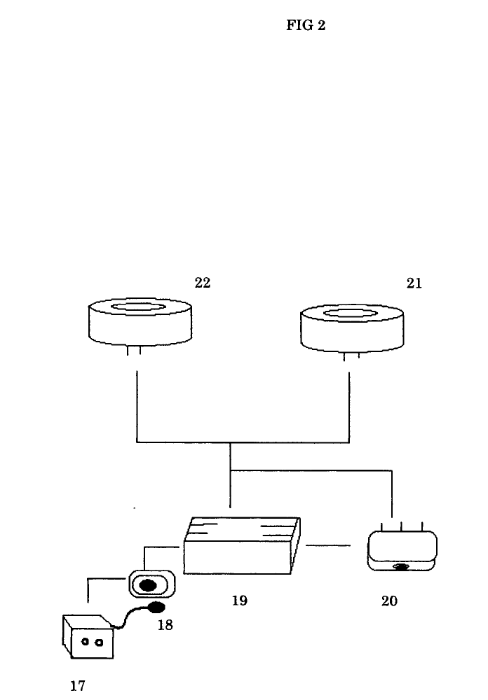

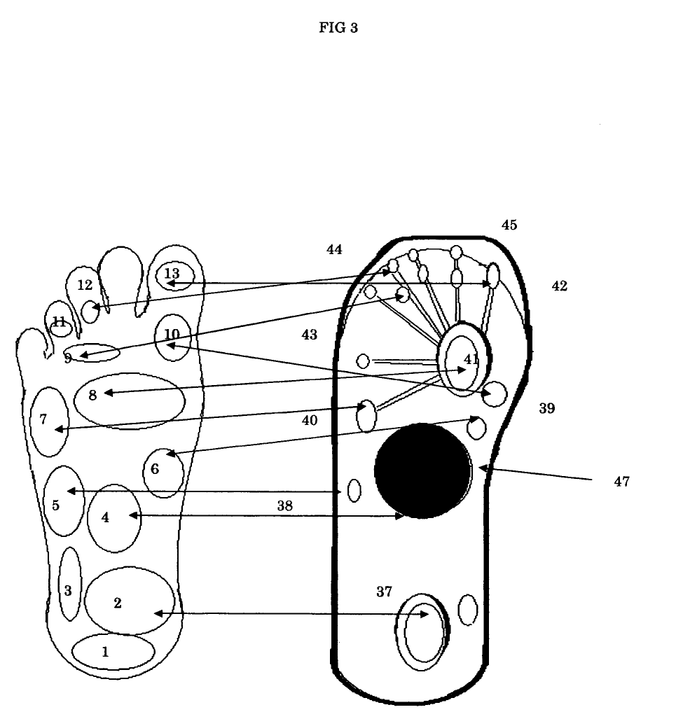

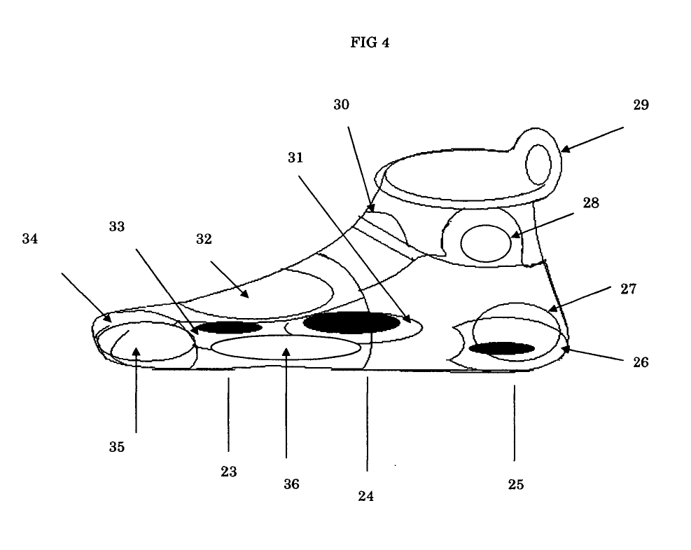

This device combines the use of innovative technology with ergonomic processes. It has been created as an alternative solution for correcting the long term effects of repetitious motion work and utilizing various types of innovative technological apparatuses. The device is a therapeutic good that has the ability to provide assistance for people with musculoskeletal disorders, occupational overuse syndrome, fatigue, and injuries related to the vascular system and the nervous system. It has been ergonomically created to help assist and relax muscle problems that occur in the most problematic regions of the foot and to stimulate the nerves throughout designated areas of the human anatomy. The invention is an ergonomic footwear device worn when in a relaxed state to rejuvenate fatigue and injury within the foot and designated areas of the human anatomy. The support system, shown in FIG. 1, FIG. 2, and FIG. 3, are connected together within this device to create oscillation for agitate fluid cavity system FIG. 4 within the invention. Agitations of the fluid cavity system, induced by the oscillation motors, create rhythmic currents throughout the mass of the footwear and have the ability to enhance blood flow and oxygen to at least the ankle and foot area.

The enhancement of blood flow and oxygen throughout areas of the foot allow fatigued muscles, ligaments, and connective tissues to recuperate at a faster rate. Statistics indicate that the most occurring injuries seen by medical physicians are the neck, elbow, heel pain, lower back pain, shoulder pain, wrist and hand pain, fore foot pain, ankle pain, and foot pain. Nerve endings located at the lower extremities of the foot have the ability to affect various sections of the body's anatomy. The circular ridge arches 37, 38,39,40,41,42,43,44, and 45 of the invention stimulate nerve sensor endings that can affect problems that may occur throughout the entire anatomy of the body. These circular ridges seen in FIG. 3 invoke nerve sensors in 1 lower back/hip/knee/ankle/foot, 2 small intestines, 3 colon, 4 kidney, 5 gall bladders/spleen, 6 adrenal glands/pancreas, 7 arms/shoulders, 8 lungs, 9 eyes/ears, 10 thyroids/parathyroid, 11 neck, 12 sinus, and 13 head/brain.

### DESCRIPTION OF THE PREFERRED EMBODIMENT

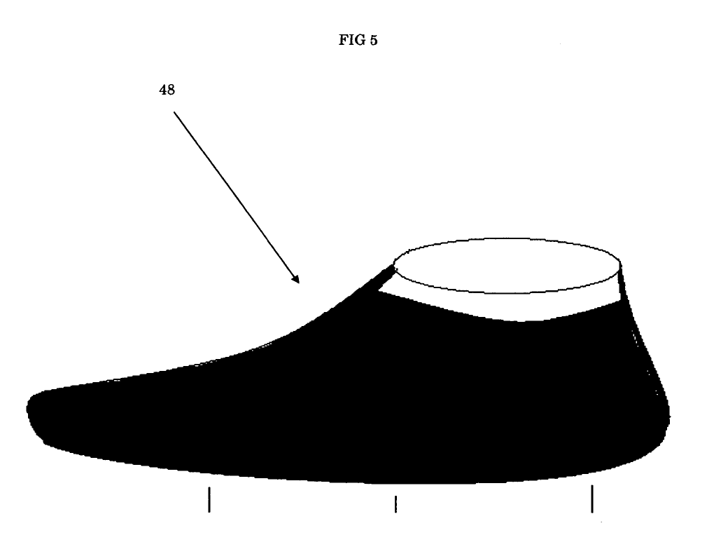

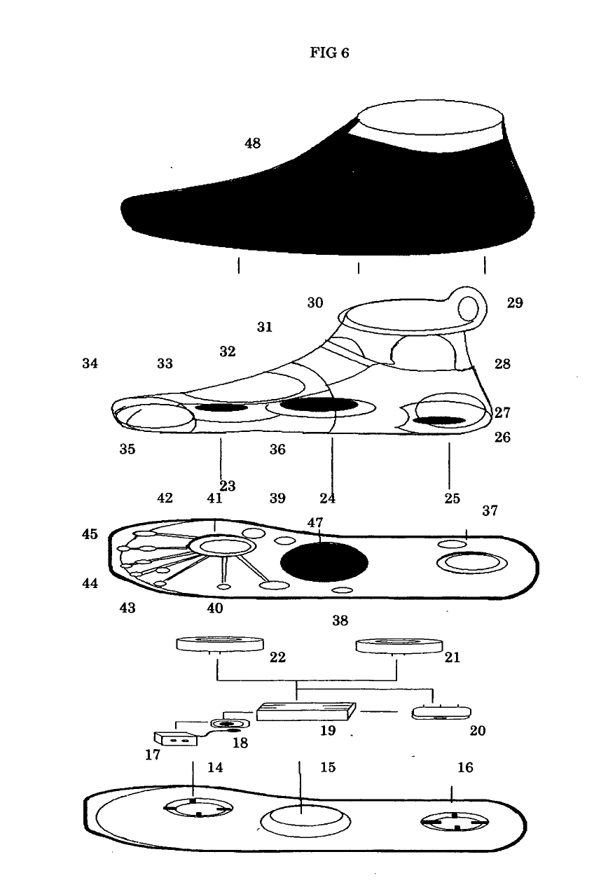

The invention is made up of five separate and unique parts that have been ergonomically designed and molded to accommodate the curves, arches and imbalanced areas of the entire foot area, seen in FIG. 6. FIG. 1, FIG. 3, FIG. 4, and FIG. 5 are made from various recyclable plastics, vinyl, silicon, and rubber materials to provide the invention with flexibility in designated areas within the footwear.

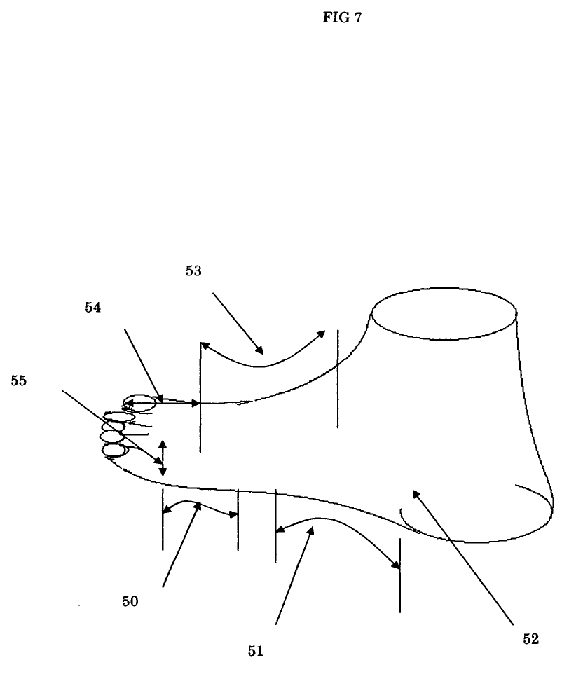

The bottom closure seen in FIG. 1 displays two ports, 14 and 16. Port 14 is placed in the vamp region under the ball area, which is indicated as 50 of FIG. 7. Port 16 is placed in the back arch area of 52. Inside of ports 14 and 16 are special grids/clamps that hold the oscillation motor systems 21 and 22 of FIG. 2. Located in the center of the bottom covering is the major circular arch bubble 15 that extends through holes 24 of FIG. 4 and 47 of FIG. 3. The circular arch bubble 15 is shaped to hold all individual parts necessary securely. Arch bubble 15 has been placed in the arch area 51 of the foot to provide support for the instep 53, seen on FIG. 7. FIG. 2 is an agitation system that consists of a power source 18, a rechargeable source 19, adjustable gauge 20, and an adapter 17 that has the ability to be connected for recharging. The agitation system is enclosed within the top casing of FIG. 3 and bottom casing FIG. 1. FIG. 3 encompasses arch bubbles 37, 38,39,40,41,42,43,44, and 45 are able to press against specified nerve endings located at the lower extremities of the foot. FIG. 1, FIG. 2, FIG. 3, and FIG. 4 fit securely together to create the completed mass of the agitation system seen in FIG. 6. FIG. 4 of the invention contains ergonomically designed fluid cavity bags created to comfortably cover the most vulnerable areas of the foot, seen in FIG. 7. The bags cover areas of the arch 51, profile 55, instep 53, and vamp 54 of the foot. Inside of the ergonomic fluid cavity bags is a propylene glycol formula that has the ability to maintain liquid form at both boiling point and freezing temperatures. When at freezing temperatures the ergonomic fluid bags have the ability to decrease swelling related injury or problematic disorder. At warmer temperatures the fluid bags are able to provide relaxation to the musculoskeletal system and induce blood flow throughout the foot area. The propylene glycol formula is a non-toxic antifreeze formula that is safer for people, pets and wildlife if a spill, puncture, leak or accident occurs. FIG. 4 has two holes that allow arch bubbles 41, and 37 to fit securely together with FIG. 3. The invention is covered by an elastic plastic material to facilitate protection for the upper extremity areas of the foot, which is seen in 48 of FIG. 5.

1. The invention contains a propylene glycol formula that is able to maintain its liquid state at low freezing temperature (−54 degrees Fahrenheit) and high boiling temperatures (260 degrees Fahrenheit). The non-toxic formula makes it safer for people, pets, and wildlife in the environment. The propylene glycol formula reduces the risk of accidental injury that can occur from spills, leaks, ruptures or misuse of the invention.

2. The invention contains fluid cavities that have the ability to be chilled and heated. When the invention is at a chilled state the fluid cavities are able to reduce swelling for rejuvenation of musculoskeletal damage, injuries, or disorders that occur in at least the ankle and foot area. When the invention is at a heated state it has the ability to relax the muscles within the foot and enhance blood flow.

3. The agitation of the fluid cavity system caused by the oscillation support system enhances the healing process of the musculoskeletal system and creates a sensation of relaxation/comfort for the foot area.

4. The agitation of the fluid cavity system enhances blood flow in at least the ankle and lower extremity of the foot.
